(19) United States
(12) Patent Application Publication
(10) Pub. No.: US 2008/0069860 A1
(43) Pub. Date: Mar. 20, 2008

(54) HYPERPHOSPHATEMIA IN DOMESTIC ANIMALS: COMPOSITIONS AND METHODS OF TREATMENT

(76) Inventors: Allison Wren, Reno, NV (US); Roy Graham, Incline Village, NV (US); Randy McElreath, Reno, NV (US); Michael Molnar, Summerfield, NC (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/465,445

(22) Filed: Aug. 17, 2006

Related U.S. Application Data
(60) Provisional application No. 60/709,179, filed on Aug. 17, 2005.
(60) Provisional application No. 60/721,717, filed on Sep. 29, 2005.

(51) Int. Cl. A61K 47/00; A21D 2/00; A23L 1/00; A23L 1/10; A23L 1/31; A23L 1/315; A23L 1/317; A61P 7/00; A23L 1/32; A23L 1/325; A23L 1/326; A23L 1/36; A61K 31/28

(52) U.S. Cl. 424/439; 426/614; 426/615; 426/618; 426/622; 426/629; 426/641; 426/643; 426/644; 426/646; 514/492

(57) ABSTRACT

The present invention is generally related to the treatment of hyperphosphatemia in domestic animals. It is specifically directed to compositions containing phosphate binders that are palatable to domestic animals and methods using such compositions. In a composition aspect, the present invention provides a composition that includes: a rare earth compound (e.g., lanthanum oxycarbonate or lanthanum carbonate hydroxide), calcium salts (e.g, calcium carbonate or calcium acetate), aluminum salts (e.g., aluminum hydroxide or a hydrophilic exchange resin; and an ingredient of domestic animal food, wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

### CROSS-REFERENCE TO RELATED APPLICATIONS

This application claims priority to U.S. Provisional Patent Application Ser. No. 60/709,179 filed on Aug. 17, 2005 and U.S. Provisional Patent Application Ser. No. 60/721,717 filed on Sep. 29, 2005, the entire disclosures of which are incorporated by reference.

### FIELD OF THE INVENTION

The present invention is generally related to the treatment of hyperphosphatemia in domestic animals. It is specifically directed to compositions containing phosphate binders that are palatable to domestic animals and methods using such compositions.

### BACKGROUND OF THE INVENTION

For patients who have renal failure, hyperphosphatemia is a significant problem. Conventional dialysis fails to reduce levels of phosphate in the blood; phosphate levels rise; and, detrimental effects from phosphate toxicity result. This phenomenon is not restricted to human patients. Large numbers of domestic animals also experience renal failure and the ensuing hyperphosphatemia.

A number of phosphate binders have been reported. These compounds, when ingested by humans experiencing hyperphosphatemia, absorb phosphate in the digestive tract. Accordingly, free phosphate is bound, the bound form is excreted, and the effects of hyperphosphatemia are reduced or eliminated.

The phosphate binders are typically provided as solid capsules or tablets. For certain types of binders, the recommended dose is substantial, which means the corresponding solid dosage form is relatively large. This can make ingestion by a human patient trying; forced administration to a domestic animal in such a circumstance can be incredibly difficult.

Providing a more ingestible form of a phosphate binder would help alleviate the difficulties associated with administering the compound to a domestic animal. A significant problem, however, arises with respect to palatability: Palatability of an ingestible substance is influenced by the formula, ingredient quality and the mouth feel (including the size, texture and shape) of the substance. One simply cannot a priori predict whether a substance will be acceptable to a domestic animal as an ingestible food.

In view of the seriousness of hyperphosphatemia, there is accordingly a need for a phosphate binder in a form that is palatable to domestic animals. That is an object of the present invention.

### SUMMARY OF THE INVENTION

The present invention is generally related to the treatment of hyperphosphatemia in domestic animals. It is specifically directed to compositions containing phosphate binders that are palatable to domestic animals and methods using such compositions.

In a composition aspect, the present invention provides a composition that includes: a rare earth compound (e.g., lanthanum oxycarbonate or lanthanum carbonate hydroxide), a calcium salt (e.g., calcium carbonate or calcium acetate), an aluminum salt (e.g., aluminum hydroxide) or a hydrophilic exchange resin; and an ingredient of domestic animal food, wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

In a kit aspect, the present invention provides a kit for the treatment of hyperphosphatemia in a domestic animal. The kit includes: a container of a composition comprising a phosphate binding compound; and, instructions related to how the compound should be added to domestic animal food to optimize animal health.

In another kit aspect, the present invention provides a kit for the treatment of hyperphosphatemia in a domestic animal. The kit includes: a container of a composition comprising a phosphate binder and domestic animal food; and, instructions related to how the composition should be used to optimize animal health.

In a method aspect, the present invention provides a method of treating hyperphosphatemia in a domestic animal. The method includes the following step of providing a domestic animal with a composition. The composition includes: a rare earth compound (e.g., lanthanum oxycarbonate or lanthanum carbonate hydroxide), a calcium salt (e.g., calcium carbonate or calcium acetate), an aluminum salt (e.g., aluminum hydroxide) or a hydrophilic exchange resin; and, an ingredient of domestic animal food, wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

In another method aspect, the present invention provides a method for treating hyperphosphatemia in a domestic animal. The method includes the steps of: mixing a phosphate binding compound with domestic animal food; and, providing the mixture to a domestic animal in an ingestible form.

### BRIEF DESCRIPTION OF THE DRAWINGS

FIG. 1 shows an X-ray diffraction scan of a compound made according to Example 1.

FIG. 2 shows an X-ray diffraction scan of a compound made according to Example 2.

### DETAILED DESCRIPTION OF THE INVENTION

The compositions of the present invention contain at least one phosphate binding compound of any suitable structure. Rare earth compounds (e.g., lanthanum oxycarbonate or lanthanum carbonate hydroxide), calcium salts (e.g, calcium carbonate or calcium acetate), aluminum salts (e.g., aluminum hydroxide) and hydrophilic anion exchange resins are typical classes of included compounds.

Where the compound is a rare earth compound, it is usually a lanthanum carbonate, lanthanum carbonate hydroxide or lanthanum oxycarbonate. Lanthanum carbonates are of the structure La₂(CO₃)₃·x H₂O, where 1≦x≦8. Preferred lanthanum carbonates are of the structure La₂(CO₃)₃ ·x H₂O, where 3≦x≦6, more preferably 3.5≦x≦5, and most preferably 3.8≦x≦4.5. Such compounds are discussed in U.S. Pat. No. 5,968,976, which is hereby incorporated-by-reference for all purposes.

Lanthanum oxycarbonates may be hydrated or anhydrous. A typical hydrated lanthanum oxycarbonate is La₂O(CO₃)₂·x H₂O, where 1≦x≦3; a typical anhydrous lanthanum oxycarbonate is La₂O₂CO₃. Such compounds are discussed in U.S. Pat. Appl. 2004161474, which is hereby incorporated-by-reference for all purposes.

Lanthanum carbonate hydroxides may be hydrated or anhydrous. A typical anhydrous lanthanum carbonate hydroxide is LaCO₃OH.

At the physiological pH of stomach of a cat and dog, around 3.0 the lanthanum oxycarbonates and lanthanum carbonate hydroxides exhibit a phosphate binding capacity of at least 300 mg of phosphate per gram of lanthanum compound. Most desirably, the lanthanum oxycarbonates exhibit a phosphate binding capacity of at least 400 mg PO₄/g of lanthanum compound. At the physiological pH of the upper small intestine of the cat or dog, around 8.0, the lanthanum oxycarbonates still bind as much as 20 mg phosphate/g lanthanum compound.

Hydrophilic anion exchange resins included in the compositions of the present invention are typically aliphatic amine polymers. The “amine” group can be present in the form of a primary, secondary or tertiary amine, quaternary ammonium salt, amidine, guanadine, hydrazine, or combinations thereof. The amine can be within the linear structure of the polymer (such as in polyethylenimine or a condensation polymer of a polyaminoalkane, e.g. diethylenetriamine, and a crosslinking agent, such as epichlorohydrin) or as a functional group pendant from the polymer backbone (such as in polyallylamine, polyvinylamine or poly(aminoethyl)acrylate). Such compounds are discussed in U.S. Pat. No. 6,858,203, which is hereby incorporated-by-reference for all purposes.

In one aspect, the polymer is characterized by a repeating unit having the formula:

—[CH₂CH(CH₂NR₂)]ₙ—

or a copolymer thereof, wherein n is an integer and each R, independently, is H or a substituted or unsubstituted alkyl, such as a lower alkyl (e.g., having between 1 and 5 carbon atoms, inclusive), alkylamino (e.g., having between 1 and 5 carbons atoms, inclusive, such as ethylamino) or aryl (e.g., phenyl) group.

In a second aspect, the polymer is characterized by a repeating unit having the formula:

—[CH₂CH(CH₂NR₃X)]ₙ—

or a copolymer thereof wherein n is an integer, each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl (e.g., phenyl) group, and each X is an exchangeable negatively charged counterion.

One example of a copolymer according to the second aspect of the invention is characterized by a first repeating unit having the formula:

—[CH₂CH(CH₂NR₃X)]ₙ—

wherein n is an integer, each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl), and each X is an exchangeable negatively charged counterion; and further characterized by a second repeating unit having the formula:

—[CH₂CH(CH₂NR₂)]ₙ—

wherein each n, independently, is an integer and each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl).

In a fourth aspect, the polymer is characterized by a repeating unit having the formula:

—[N(R)CH₂CH₂]ₙ—

or a copolymer thereof, wherein n is an integer, and R is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl).

One example of a copolymer according to the second aspect of the invention is characterized by a first repeating unit having the formula:

—[N(R)CH₂CH₂]ₙ—

wherein n is an integer, and R is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl); and further characterized by a second repeating unit having the formula:

—[N(X)(H)(R)CH₂CH₂]ₙ—

wherein each n, independently, is an integer and R is H or a substituted or unsubstituted alkyl (e.g., having between 1 and 5 carbon atoms, inclusive), alkylamino (e.g., having between 1 and 5 carbon atoms, inclusive, such as ethylamino) or aryl group (e.g., phenyl).

In a fifth aspect, the polymer is characterized by a repeating group having the formula:

—[N(X)(R₁)(R₂)CH₂CH₂]ₙ—

or a copolymer thereof, wherein n is an integer, and each R₁ and R₂, independently, is H or a substituted or unsubstituted alkyl (e.g., having between 1 and 5 carbon atoms, inclusive), and alkylamino (e.g., having between 1 and 5 carbons atoms, inclusive, such as ethylamino) or aryl group (e.g., phenyl), and each X is an exchangeable negatively charged counterion.

In one preferred polymer according to the fifth aspect of the invention, at least one of the R groups is a hydrogen atom.

In a sixth aspect, the polymer is characterized by a repeat unit having the formula:

—[CH(NR₂R₂)CH₂]ₙ—

or a copolymer thereof, where n is an integer, each R₁ and R₂, independently, is H, a substituted or unsubstituted alkyl group containing 1 to 20 carbon atoms, an alkylamino group (e.g., having between 1 and 5 carbons atoms, inclusive, such as ethylamino), or an aryl group containing 6 to 12 atoms (e.g., phenyl).

In a seventh aspect, the polymer is characterized by a repeat unit having the formula:

—[CH(NR₁R₂R₃X)CH_{2]}—

or a copolymer thereof, wherein n is an integer, each R₁, R₂ and R₃, independently, is H, a substituted or unsubstituted alkyl group containing 1 to 20 carbon atoms, an alkylamino group (e.g., having between 1 and 5 carbons atoms, inclusive, such as ethylamino), or an aryl group containing 6 to 12 atoms (e.g., phenyl), and each X is an exchangeable negatively charged counterion.

In each case, the R groups can carry one or more substituents. Suitable substituents include therapeutic anionic groups, e.g., quaternary ammonium groups, or amine groups, e.g., primary and secondary alkyl or aryl amines. Examples of other suitable substituents include hydroxy, alkoxy, carboxamide, sulfonamide, halogen, alkyl, aryl, hydrazine, guanadine, urea, and carboxylic acid esters, for example.

The polymers are preferably crosslinked, in some cases by adding a crosslinking agent to the reaction mixture during or after polymerization. Examples of suitable crosslinking agents are diacrylates and dimethacrylates (e.g., ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate, polyethyleneglycol diacrylate), methylene bisacrylamide, methylene bismethacrylamide, ethylene bisacrylamide, epichlorohydrin, epibromohydrin, toluene diisocyanate, ethylenebismethacrylamide, ethylidene bisacrylamide, divinyl benzene, bisphenol A dimethacrylate, bisphenol A diacrylate, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, acryloyl chloride, or pyromellitic dianhydride.

The amount of crosslinking agent is typically between about 0.5 and about 75 weight %, and preferably between about 1 and about 25% by weight, based upon the combined weight of crosslinking and monomer. In another embodiment, the crosslinking agent is present between about 2 and about 20% by weight of polymer.

In some cases the polymers are crosslinked after polymerization. One method of obtaining such crosslinking involves reaction of the polymer with difunctional crosslinkers, such as epichlorohydrin, succinyl dichloride, the diglycidyl ether of bisphenol A, pyromellitic dianhydride, toluence diisocyanate, and ethylenediamine. A typical example is the reaction of poly(ethyleneimine) with epichlorohydrin. In this example the epichlorohydrin (1 to 100 parts) is added to a solution containing polyethyleneimine (100 parts) and heated to promote reaction. Other methods of inducing crosslinking on already polymerized materials include, but are not limited to, exposure to ionizing radiation, ultraviolet radiation, electron beams, radicals, and pyrolysis.

Examples of preferred crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride.

The compositions of the present invention are primarily intended to treat hyperphosphatemia in domestic animals. Exemplary domestic animals include dogs, cats, horses, rabbits, cows, goats and pigs. The present invention is particularly directed to the treatment of dogs, cats and horses.

Typical dog foods contain a protein source, such as chicken, beef, lamb, chicken meal or lamb meal. Preservatives, e.g., tocopherols, BHT and BHA, are also usual ingredients. Other ingredients may include corn, rice and bone meal. Typical cat foods may include, for example, fish meal, fish, egg product, beef, chicken, rice, corn gluten meal, poultry by-product meal, wheat flour, beef tallow, and corn. Horse food often contains ingredients such as maple syrup, honey, apple, flaxseed, flaxseed meal, rice bran and germ, oats, barley, corn, and wheat bran.

Compositions of the present invention typically include a phosphate binder in combination with domestic animal food. The combination may take any suitable form. For instance, it may be in the form of particles, grains, pellets, etc. that contain the phosphate binder and the domestic animal food. Alternatively, it may be in the form of a simple physical admixture of the components, e.g., mixing the phosphate binder with the domestic animal food. Another form would involve sprinkling a composition including the phosphate binder onto domestic animal food. One could then mix it before serving it to the animal.

The following are exemplary compositions of the present invention:

1. A particle, grain or pellet including a lanthanum oxycarbonate or lanthanum carbonate hydroxide and domestic animal food including at least one of the following ingredients: chicken, beef, lamb, chicken meal or lamb meal, tocopherols, BHT and BHA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

2. A particle, grain or pellet including a lanthanum carbonate and domestic animal food including at least one of the following ingredients: chicken, beef, lamb, chicken meal or lamb meal, tocopherols, BET and BEA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

3. A particle, grain or pellet including an aliphatic amine polymer and domestic animal food including at least one of the following ingredients: chicken, beef, lamb, chicken meal or lamb meal, tocopherols, BHT and BHA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

4. A physical admixture including a lanthanum oxycarbonate or lanthanum carbonate hydroxide and domestic animal food including at least one of the following ingredients: chicken, beef, lamb, chicken meal or lamb meal, tocopherols, BHT and BHA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

5. A physical admixture including a lanthanum carbonate and domestic animal food including at least one of the following ingredients: chicken, beef, lamb, chicken meal or lamb meal, tocopherols, BHT and BHA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

6. A physical admixture including an aliphatic amine polymer and domestic animal food including at least one of the following ingredients: chicken, beef, lamb, chicken meal or lamb meal, tocopherols, BHT and BHA, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

When lanthanum oxycarbonate or lanthanum carbonate hydroxide is administered as the phosphate binder, the amount of administered to the domestic animal during a single administration typically ranges from 1.0 to 100 mg/kg body weight. Oftentimes the amount ranges from 30.0 to 80 mg/kg body weight. In certain cases the amount of administered lanthanum oxycarbonate ranges from 40.0 to 75.0 mg/kg body weight.

The kits of the present invention are of two general types. The first includes a container (e.g., bag, jar, can, etc.) of a composition comprising a lanthanum binding compound and instructions related to how the compound should be added to domestic animal food. Information such as the amount of phosphate binder to include, the regimen for feeding the domestic animal, and the type of domestic animal food it can be added to is typically included on the instructions.

The second includes a container of a composition of the present invention (i.e., lanthanum binding compound in combination with domestic animal food) and instructions related to how the composition should be used. Typical information included in the instructions is the amount of food to be served to the domestic animal and regimen for providing the food (e.g., time during the day and number of times provided per day).

The methods of the present invention are of two general types. The first method includes at least the following step: providing a domestic animal with a composition of the present invention in an ingestible form. The second method includes at least the following steps: 1) mixing a lanthanum binding compound with domestic animal food; and, 2) providing the mixture to a domestic animal in an ingestible form.

### EXAMPLES

### Example 1

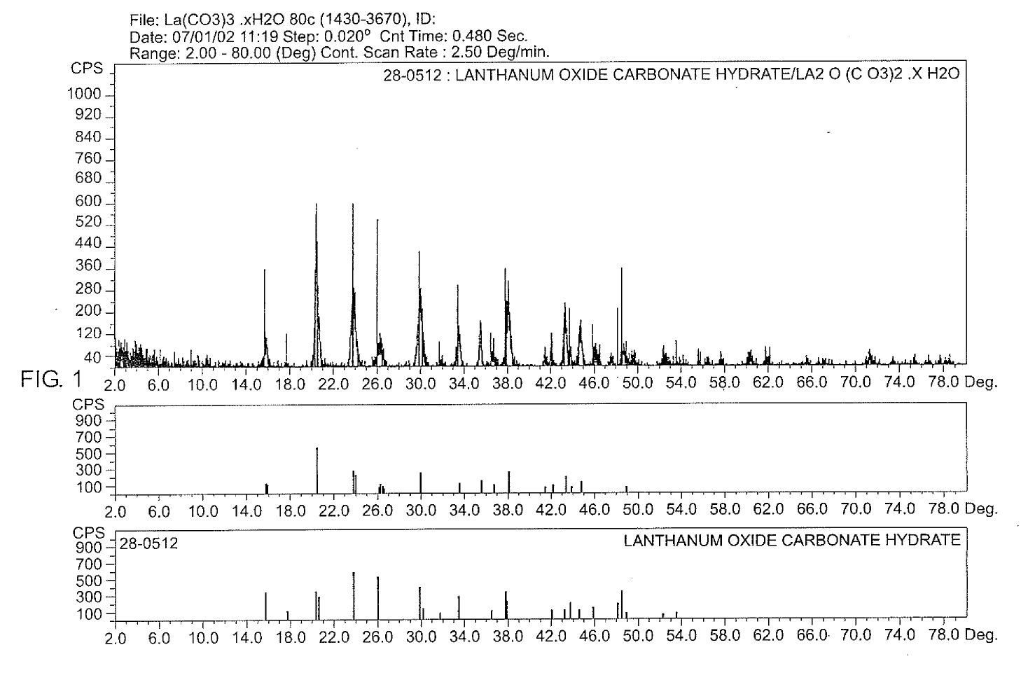

An aqueous HCl solution having a volume of 334.75 ml and containing LaCl₃ (lanthanum chloride) at a concentration of 29.2 wt % as La₂O₃ was added to a four liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper, the filtrate was separated from the white powder product. The filter cake was mixed four times with 2 liters of distilled water and filtered to wash away the NaCl formed during the reaction. The washed filter cake was placed into a convection oven set at 105° C. for 2 hours, or until a stable weight was observed. The product consists of lanthanum carbonate hydroxide. An X-ray diffraction scan of the compound, as compared to a standard sample, is shown in FIG. 1.

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Lanthanum carbonate hydroxide powder made as described above was added to the solution. The amount of lanthanum carbonate hydroxide powder was such that the amount of La in suspension was 3 times the stoichiometric amount needed to react completely with the phosphate. Samples of the suspension were taken at time intervals through a filter that separated all solids from the liquid. The liquid sample was analyzed for phosphorous.

### Example 2

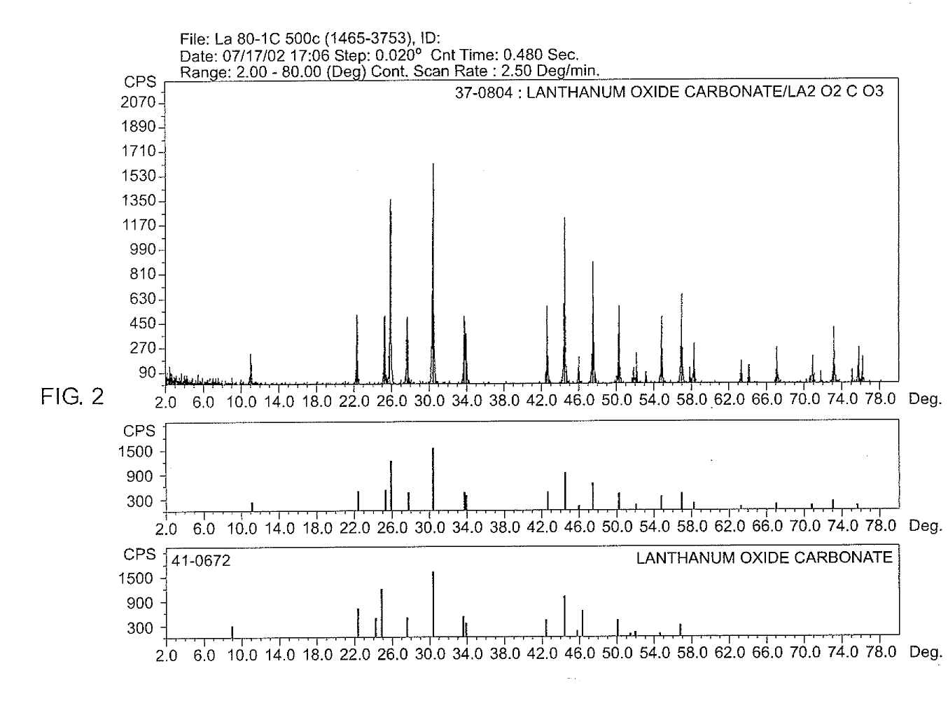

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times with 2 liters of distilled water and filtered to wash away the NaCl formed during the reaction. The washed filter cake was placed into a convection oven set at 105° C. for 2 hours until a stable weight was observed. Finally, the lanthanum oxycarbonate was placed in an alumina tray in a muffle furnace. The furnace temperature was ramped to 500° C. and held at that temperature for 3 hours. The resultant product was determined to be anhydrous lanthanum oxycarbonate La₂O₂CO₃. An X-ray diffraction scan of the compound, as compared to a standard, is shown in FIG. 2.

The process was repeated three times. In one case, the surface area of the white powder was determined to be 26.95 m²/gm. A micrograph shows that the structure in this compound is made of equidimensional or approximately round particles of about 100 nm in size. An X-ray diffraction pattern showed that the product made is an anhydrous lanthanum oxycarbonate written as La₂O₂CO₃.

To determine the reactivity of this lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Anhydrous lanthanum oxycarbonate made as described above, was added to the solution. The amount of anhydrous lanthanum oxycarbonate was such that the amount of La in suspension was 3 times the stoichiometric amount needed to react completely with the phosphate. Samples of the suspension were taken at intervals, through a filter that separated all solids from the liquid.

### Example 3

A solution containing 100 g/l of La as lanthanum acetate is injected in a spray-drier with an outlet temperature of 250° C. The intermediate product corresponding to the spray-drying step is recovered in a bag filter. This intermediate product is calcined at 600° C. for 4 hours. X-Ray diffraction of the product showed that it consists of anhydrous lanthanum oxycarbonate. The formula for this compound is written as (La₂CO₅).

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. La₂CO₅ powder, made as described above, was added to the solution. The amount of lanthanum oxycarbonate was such that the amount of La in suspension was 3 times the stoichiometric amount needed to react completely with the phosphate. Samples of the suspension were taken at intervals through a filter that separated all solids from the liquid. The liquid sample was analyzed for phosphorous.

### Example 4

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times, each with 2 liters of distilled water and filtered to wash away the NaCl formed during the reaction. The washed filter cake was placed into a convection oven set at 105° C. for 2 hours or until a stable weight was observed. The X-Ray diffraction pattern of the product showed that it consists of lanthanum carbonate hydroxide. The surface area of the product was determined by the BET method.

### Example 5

In Vivo Study in Rats

Groups of six adult Sprague-Dawley rats underwent 5/6th nephrectomy in two stages over a period of 2 weeks and were then allowed to recover for a further two weeks prior to being randomized for treatment. The groups received vehicle (0.5% w/v carboxymethyl cellulose), or lanthanum oxycarbonate suspended in vehicle, once daily for 14 days by oral lavage (10 ml/kg/day). The dose delivered 314 mg elemental lanthanum/kg/day. Dosing was carried out immediately before the dark (feeding) cycle on each day. Urine samples (24 hours) were collected prior to surgery, prior to the commencement of treatment, and twice weekly during the treatment period. Volume and phosphorus concentration were measured.

Feeding—During the acclimatization and surgery period, the animals were given Teklad phosphate sufficient diet (0.5% Ca, 0.3%P; Teklad No. TD85343), ad libitum. At the beginning of the treatment period, animals were pair fed based upon the average food consumption of the vehicle-treated animals the previous week.

5/6 Nephrectomy—After one week of acclimatization, all animals were subjected to 5/6 nephrectomy surgery. The surgery was performed in two stages. First, the two lower branches of the left renal artery were ligated. One week later, a right nephrectomy was performed. Prior to each surgery, animals were anesthetized with an intra-peritoneal injection of ketamine/xylazine mixture (Ketaject a 100 mg/ml and Xylaject at 20 mg/ml) administered at 10 ml/kg. After each surgery, 0.25 mg/kg Buprenorphine was administered for relief of post-surgical pain. After surgery, animals were allowed to stabilize for 2 weeks to beginning treatment.

Results show a decrease in phosphorus excretion, a marker of dietary phosphorus binding, after administration of the lanthanum oxycarbonate or lanthanum carbonate hydroxide (at time>0), compared to untreated rats.

### Example 6

Dog Study

Six adult beagle dogs were dosed orally with capsules of lanthanum oxycarbonate LaCO₃OH (compound A) or La₂O₂CO₃ (compound B) in a cross-over design using a dose of 2250 mg elemental lanthanum twice daily (6 hours apart). The doses were administered 30 minutes after provision of food to the animals. At least 14 days washout was allowed between the crossover arms. Plasma was obtained pre-dose and 1.5, 3, 6, 7.5, 9, 12, 24, 36, 48, 60, and 72 hours after dosing and analyzed for lanthanum using ICP-MS. Urine was collected by catheterization before and approximately 24 hours after dosing and creatinine and phosphorus concentrations measured The tests led to reduction of urine phosphate excretion, a marker of phosphorous binding.

### Example 7

Palatability Study

Lanthanum oxycarbonate was mixed with wet and dry dog food and presented to 9 different dogs, almost all over 40 pounds. Each of the dogs ate the mixture, although 2 hesitated for some hours before eating. None of the dogs exhibited signs of nausea, vomiting, bloating or flatus during hours post meal.

Lanthanum oxycarbonate was mixed with cat food and presented to 2 cats, both old and one overweight. The first cat ate the food mixture. The second, which was the overweight cat, did not eat the mixture.

1. A composition, wherein the composition comprises:
a) a rare earth compound or a hydrophilic exchange resin; and,
b) an ingredient of domestic animal food, wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

2. The composition according to claim 1, wherein the composition comprises a rare earth compound, and wherein the rare earth compound is a lanthanum oxycarbonate or lanthanum carbonate hydroxide.

3. The composition according to claim 1, wherein the composition comprises a rare earth compound, and wherein the rare earth compound is a lanthanum carbonate.

4. The composition according to claim 1, wherein the composition comprises a hydrophilic exchange resin, and wherein the hydrophilic exchange resin is an aliphatic amine polymer.

5. The composition according to claim 1, wherein the composition comprises calcium acetate or calcium carbonate

6. The composition according to claim 1, wherein the composition comprises aluminium hydroxide

7. The composition according to claim 2, wherein the composition is in the form of a particle, grain or pellet.

8. A kit for the treatment of hyperphosphatemia in a domestic animal, wherein the kit comprises:
a) a container of a composition comprising a phosphate binding compound; and,
b) instructions related to how the compound should be added to domestic animal food to optimize animal health.

9. The kit according to claim 8, wherein the phosphate binding compound is a lanthanum oxycarbonate or lanthanum carbonate hydroxide.

10. The kit according to claim 9, wherein the instructions include information regarding the amount of lanthanum oxycarbonate to include with the domestic animal food and a regimen for feeding the domestic animal.

11. The kit according to claim 9, wherein the phosphate binding compound is lanthanum carbonate hydroxide.

12. The kit according to claim 10, wherein the lanthanum oxycarbonate is La₂O₂CO₃.

13. A kit for the treatment of hyperphosphatemia in a domestic animal, wherein the kit comprises:
a) a container of a composition comprising a phosphate binder and domestic animal food; and,
b) instructions related to how the composition should be used to optimize animal health.

14. The kit according to claim 13, wherein the domestic animal food comprises an ingredient, and wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

15. The kit according to claim 14, wherein the phosphate binder is lanthanum carbonate hydroxide.

16. The kit according to claim. 14, wherein the phosphate binder is La₂O₂CO₃.

17. The kit according to claim 15, wherein the instructions include information regarding the amount of food to be served to the domestic animal and a regimen for providing the food.

18. A method for treating hyperphosphatemia in a domestic animal, wherein the method comprises the step of providing a domestic animal with a composition, and wherein the composition comprises:
a) a rare earth compound or a hydrophilic exchange resin; and,
b) an ingredient of domestic animal food, wherein the ingredient is selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

19. The method according to claim 18, wherein the composition comprises lanthanum carbonate hydroxide.

20. The method according to claim 18, wherein the composition comprises La₂O₂CO₃.

21. The method according to claim 18, wherein the composition comprises La₂(CO₃)₃ ·×H₂O, where 1≦x≦8.

22. The method according to claim 18, wherein the composition comprises an aliphatic amine polymer.

23. A method for treating hyperphosphatemia in a domestic animal, wherein the method comprises the steps of:
a) mixing a phosphate binding compound with domestic animal food; and,
b) providing the mixture to a domestic animal in an ingestible form.

24. The method according to claim 23, wherein the domestic animal food comprises ingredients, and wherein the ingredients are selected from a group consisting of chicken, beef, lamb, chicken meal or lamb meal, corn, rice, bone meal, fish meal, fish, egg product, beef, beef meal, corn gluten meal, poultry by-product meal, wheat flour, beef tallow, maple syrup, honey, apple, flaxseed, flaxseed meal, rice bran and germ, oats, barley, and wheat bran.

25. The method according to claim 24, wherein the phosphate binding compound is lanthanum carbonate hydroxide.

26. The method according to claim 24, wherein the phosphate binding compound is La₂O₂CO₃.

27. The method according to claim 24, wherein the phosphate binding compound is La₂(CO₃)₃·×H₂O, where 1≦x≦8.
